(19) United States
(12) Patent Application Publication
(10) Pub. No.: US 2020/0338244 A1
(43) Pub. Date: Oct. 29, 2020

(54) APPARATUS FOR NEGATIVE PRESSURE WOUND THERAPY

(71) Applicants: S2MEDICAL AB, Linkoping (SE); ABSORBEST AB, Kisa (SE)

(72) Inventors: Petter SIVLER, Linkoping (SE); Marten SKOG, Nassjo (SE); Luca CONTI, Linkoping (SE); Hans KARLSSON, Halmstad (SE); Rolf ROVANIEMI, Rimforsa (SE)

(73) Assignees: S2MEDICAL AB, Linkoping (SE); ABSORBEST AB, Kisa (SE)

(21) Appl. No.: 16/758,257

(22) PCT Filed: Oct. 24, 2018
(86) PCT No.: PCT/IB2018/058303

§ 371 Date: Apr. 22, 2020

(30) Foreign Application Priority Data
Oct. 25, 2017 (IT) 102017000120992

(51) Int. Cl. A61M 1/00

(52) U.S. Cl. CPC A61M 1/0088; A61M 1/0096

(57) ABSTRACT

An apparatus for negative pressure wound therapy (NPWT) includes: a pump, a sealed wound cover applicable to a wound, and a tube connecting the pump to the sealed wound cover for generating a negative pressure within the sealed wound cover. The tube includes a longitudinal segment which is at least partially filled in with an absorbent material adapted to absorb wound fluids coming from the wound and travelling along the tube.

The present invention relates to an apparatus for negative pressure wound therapy (NPWT).

Negative pressure wound therapy (NPWT) is a therapeutic technique used nowadays to promote healing in acute or chronic wounds and in second or third degree burns. NPWT has proved to be beneficial also in the treatment of diabetic ulcers and in the management of open abdomen (OA) procedures, such as laparotomy.

Standard NPWT involves the controlled application of sub-atmospheric (i.e. negative) pressure to the local wound environment, using a sealed wound dressing connected to a vacuum pump by means of a tube. The dressing is applied on top of the wound and comprises an absorbent material which is in contact with the wound. The dressings used for NPWT include open-cell foam dressings and gauze, sealed with an occlusive cover, or film drape, intended to maintain a vacuum condition at the wound site.

Generally the whole wound bed is filled in with a foam dressing which can be cut in order to fit the contours of the wound. The film drape can then be applied on top of the foam dressing in order to create, around the dressing, the seal which is necessary to keep the negative pressure in the wound site.

The apparatuses for standard NPWT generally comprise a canister, or container, where the fluids drawn out from the wound are collected.

However, the present technology for NPWT has many limitations.

One of the main limitations of the standard NPWT apparatuses consists in that they require to frequently empty, clean and sanitize the canister where the wound fluids are collected.

Another drawback of the standard NPWT apparatuses regards the fact that the wound fluids risk to reach and damage the vacuum pump.

Another drawback of the standard NPWT apparatuses consists in that the tubes which are used to connect the sealed wound dressing to the vacuum pump in order to create the negative pressure in the wound site, are also passed through by the wound fluids, and therefore they require frequent changes for hygienical reasons.

A further drawback is related to the use of the absorbent material, such as a foam dressing, to be placed on the wound site. In fact, the foam dressing and the sealed cover on top of it cover the wound and prevent any kind of inspection of the wound without removing the cover and the foam material.

Furthermore, the foam dressing needs to be changed frequently since it is in constant contact with the wound. A frequent change of the foam dressing and of the cover, in particular in the case of patients with chronic wounds, results in a lot of wasted material, which needs to be properly disposed.

Furthermore, such frequent changes of the foam dressing require frequent halts of the NPWT apparatus and therefore frequent interruptions of the application of the negative pressure to the wound, which is not beneficial for the wound treatment. In particular, at each change of the sealed wound dressing, the NPWT apparatus needs to be detached from the used sealed wound dressing and attached to a new dressing, while guaranteeing the airtight seal of the new dressing.

In addition, at every dressing change the wound is exposed to external agents, thus increasing the risks of infections.

Another drawback of the standard NPWT consists in that the foam dressing tends to stick to the wound bed. The removal of the dressing can thus be difficult and also painful for the patient.

The aim of the present invention is to provide an apparatus for negative pressure wound therapy which solves the above drawbacks and ameliorates the NPWT administration.

Within this aim, an object of the present invention is to provide an apparatus for NPWT which guarantees extremely high levels of hygiene for the patient.

Another object of the invention is to provide an apparatus for NPWT which reduces the ailments for the patient and which limits the risks of wound infection.

A further object of the invention is to provide an apparatus for NPWT which is easy to use and which does not require complex procedures for guaranteeing the continuous administration of the therapy to the patient.

Another object of the invention is to provide an apparatus for NPWT which produces less waste.

A still further object of the invention is to provide an apparatus for NPWT which is capable of offering the widest guarantees of reliability and safety in use and the use of which entails also an economic competitive advantage.

This aim and these and other objects which will become better apparent hereinafter are achieved by an apparatus for negative pressure wound therapy according to claim 1.

Additional features are provided in the dependent claims.

Further characteristics and advantages of the invention will become better apparent from the detailed description of a preferred, but not exclusive, embodiment of an apparatus for negative pressure wound therapy, which is illustrated by way of a non-limiting example in the following figures:

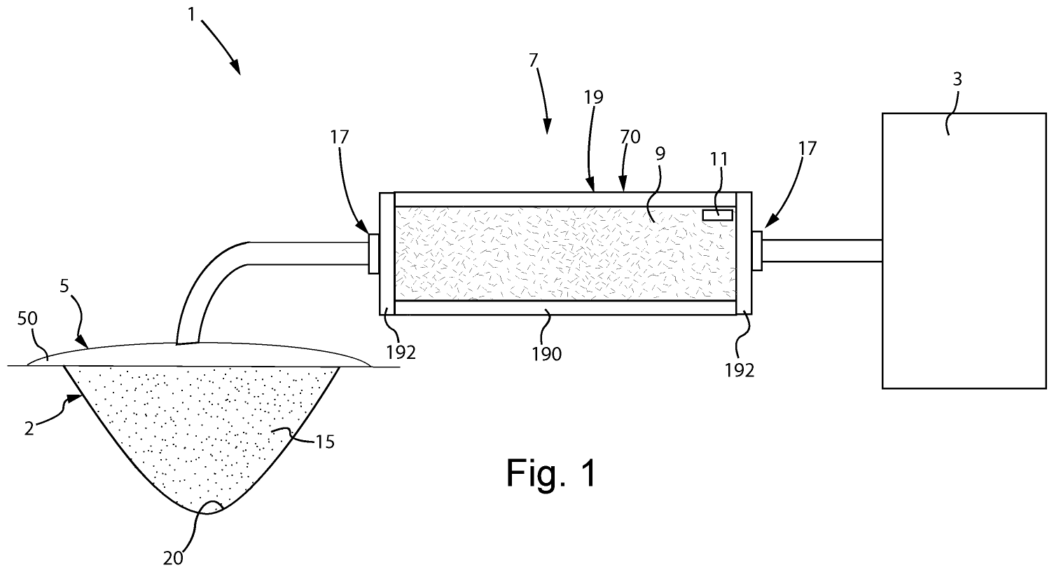

FIG. 1 is a schematic view of a first embodiment of the apparatus for NPWT according to the invention;

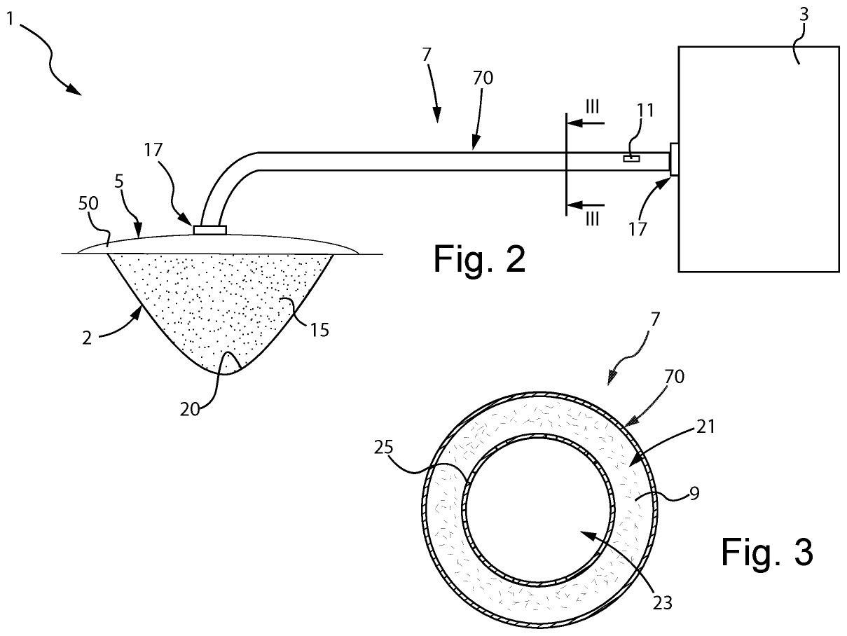

FIG. 2 is a schematic view of a second embodiment of the apparatus for NPWT according to the invention;

FIG. 3 is a sectional view of the apparatus for NPWT shown in FIG. 2, taken along the axis III-III.

With reference to the figures, the apparatus for negative pressure wound therapy, generally designated by the reference numeral 1, comprises a pump 3, a sealed wound cover 5 applicable to a wound 2, and at least a tube 7 connecting the pump 3 to the wound cover 5 for generating a negative pressure within said wound cover 5, and in particular between the wound bed 20 and the wound cover 5.

According to the invention, the tube 7 comprises at least a longitudinal segment 70 which is at least partially filled in with an absorbent material 9 adapted to absorb wound fluids coming from the wound 2 and travelling along the tube 7, in order to prevent the wound fluids from reaching the pump 3.

Advantageously the apparatus 1 can comprise a sensor 11 positioned in said longitudinal segment 70 of said tube 5 and configured to detect the saturation of the absorbent material 9 by the wound fluids.

Alternatively it is possible to limit the use in time of the apparatus 1 so that the absorbent material 9 can be replaced before getting saturated by the wound fluids.

Advantageously the wound cover 5 is constituted exclusively by a transparent adhesive film 50, which allows a direct inspection of the wound 2 and of the wound bed 20, without any need to remove the sealed wound cover 5 itself.

Alternatively, the wound cover 5 can be constituted exclusively by a non-adhesive layer, such as a plastic plate, preferably transparent, and an adhesive film adapted to hold in place the non-adhesive layer.

Advantageously the apparatus 1 does not need any dressing as such inside the wound bed 20 to absorb the wound fluids in situ.

The apparatus for NPWT can comprise a collagen filling 15 adapted to fill the wound bed 20 of the wound 2. Such collagen filling 15 is particularly adapted for deep and/or large wounds 2. The sealed wound cover 5 can be applied onto the wound 2 so as to enclose completely the collagen filling 15.

The apparatus 1 advantageously comprises detachable connecting means 17 for detaching the tube 7 or portions thereof.

For example, the apparatus 1 can comprise detachable connecting means 17 which allows to detach and replace the longitudinal segment 70 of the tube 7 where the absorbent material 9 is located.

The absorbent material 9 advantageously comprises absorbent polymers, or superabsorbent polymers, and fibrous materials. Such fibrous materials can comprise cellulose fibers and/or polymeric fibers.

Advantageously the absorbent material 9 is capable of substantially holding the fluids which have been previously absorbed, even when under load.

As shown in the first embodiment of the apparatus 1 illustrated in FIG. 1, the longitudinal segment 70 of the tube 7 consists of a sealed cartridge 19 filled in with the absorbent material 9.

The sealed cartridge 19 advantageously comprises two longitudinal ends detachably connected to the tube 7 by means of the detachable connecting means 17.

The sealed cartridge 19 can comprise a cylindrical tube 190 capped at its ends with plastic caps 192 associated to the detachable connecting means 17.

The plastic caps 192 are configured to hold the absorbent material 9 inside the cylindrical tube 190 and to keep the cartridge 19 sealed.

The sealed cartridge 19 can be replaced whenever full.

The absorbent material 9 does not significantly jeopardize the efficiency of the pump 3 in creating the negative pressure for the wound therapy, but it absorbs any wound fluid passing through the tube 7.

According to a variant of the first embodiment of the apparatus 1, only the absorbent material 9 contained inside the sealed cartridge 19 can be replaced when saturated with the wound fluids, by removing at least one of the plastic caps 192, instead of replacing the whole cartridge 19.

The replacement of the cartridge 19 or of the absorbent material 9 contained therein can be performed when the sensor 11 indicates that the cartridge 19 is full of wound fluids.

As shown in the second embodiment of the apparatus 1 illustrated in FIGS. 2 and 3, the longitudinal segment 70 of the tube 7 comprises a radially external annular portion 21 and a central portion 23 which are separated by a net 25 or by a wall comprising a plurality of through-holes.

Advantageously either the annular portion 21 or the central portion 23 can be filled in with the absorbent material 9.

The net or wall 25 advantageously comprises meshes or through-holes which are dimensioned so as to have a capillary absorption effect on the wound fluid which laps onto the net 25.

Advantageously the longitudinal segment 70 containing the absorbent material 9 can be as long as the whole tube 7. In other words, substantially the entire tube 7 can comprise the absorbent material 9.

The sensor 11 can be located in an area of the tube 7 which is close to the end of the tube which is connected to the pump 3.

The detachable connecting means 17 can be provided either at the ends of the tube 7 or at the ends of the longitudinal segment 70 where the absorbent material 9 is located.

The present invention also regards a cartridge 19 for an apparatus for negative pressure wound therapy (NPWT), such as the apparatus described above, According to the invention the cartridge 19 is filled in with an absorbent material 9 adapted to absorb wound fluids coming from a wound 2.

In practice it has been found that the apparatus for negative pressure wound therapy according to the present invention achieves the intended aim and objects in that it makes it possible to more easily manage the administration of the therapy to patients.

Another advantage of the NPWT apparatus, according to the invention, consists in that it allows to prevent the wound maceration, since the wound fluids are immediately brought away from the wound site and directed towards the absorbent material.

Another advantage of the NPWT apparatus, according to the invention, consists in that it requires less interventions on the wound itself with respect to the standard negative pressure wound therapies, which require frequent changes of the wound dressing.

A further advantage of the NPWT apparatus, according to the invention, consists in that it allows to easily inspect the wound site, possible without removing the sealed wound cover.

Another advantage of the NPWT apparatus, according to the invention, consists in that the replacement of the wound fluid absorbent material is easier, safer and more hygienic to perform as compared to standard techniques.

Still another advantage of the invention is that of reducing the exposition of the wound to external agents thus reducing the risks of infections.

The apparatus for negative pressure wound therapy thus conceived is susceptible of numerous modifications and variations, all of which are within the scope of the appended claims. Moreover, all the details may be substituted by other, technically equivalent elements. In practice the materials employed, provided they are compatible with the specific use, and the contingent dimensions and shapes, may be any according to requirements.

1. An apparatus for negative pressure wound therapy (NPWT), comprising:
a pump;
a sealed wound cover applicable to a wound; and
at least a tube connecting said pump to said sealed wound cover for generating a negative pressure within said sealed wound cover,
wherein said tube comprises at least a longitudinal segment which is at least partially filled in with an absorbent material adapted to absorb wound fluids coming from said wound and travelling along said tube.

2. The apparatus for negative pressure wound therapy (NPWT), according to claim 1, further comprising a sensor positioned in said longitudinal segment of said tube and configured to detect the saturation of said absorbent material by said wound fluids.

3. The apparatus for negative pressure wound therapy (NPWT), according to claim 1, wherein said sealed wound cover is constituted exclusively by a transparent adhesive film.

4. The apparatus for negative pressure wound therapy (NPWT), according to claim 1, further comprising a collagen filling adapted to fill a wound bed of said wound.

5. The apparatus for negative pressure wound therapy (NPWT), according to claim 1, further comprising detachable connecting means for detaching said tube or portions thereof.

6. The apparatus for negative pressure wound therapy (NPWT), according to claim 1, wherein said longitudinal segment of said tube consists of a sealed cartridge filled in with said absorbent material.

7. The apparatus for negative pressure wound therapy (NPWT), according to claim 6, wherein said sealed cartridge comprises two longitudinal ends detachably connected to said tube by means of detachable connecting means.

8. The apparatus for negative pressure wound therapy (NPWT), according to claim 1, wherein said longitudinal segment of said tube comprises a radially external annular portion and a central portion wherein said annular portion and said central portion are separated by a net and wherein said annular portion is filled in with said absorbent material.

9. The apparatus for negative pressure wound therapy (NPWT), according to claim 1, wherein said longitudinal segment of said tube comprises a radially external annular portion and a central portion wherein said annular portion and said central portion are separated by a net and wherein said central portion is filled in with said absorbent material.

10. The apparatus for negative pressure wound therapy (NPWT), according to claim 8, wherein said net comprises meshes which are dimensioned so as to have a capillary absorption effect on said wound fluid.

11. A cartridge for an apparatus for negative pressure wound therapy (NPWT), wherein said cartridge is filled in with an absorbent material adapted to absorb wound fluids coming from a wound.
